(19) United States
(12) Patent Application Publication
(10) Pub. No.: US 2009/0112211 A1
(43) Pub. Date: Apr. 30, 2009

(54) Method of bone surgery

(75) Inventor: Alan J. Johnstone, Aberdeen (GB)

Correspondence Address: EDWARDS ANGELL PALMER & DODGE LLP, P.O. BOX 55874, BOSTON, MA, 02205, US

(73) Assignee: The Robert Gordon University, Aberdeen (GB)

(21) Appl. No.: 12/077,984

(22) Filed: Mar. 24, 2008

(30) Foreign Application Priority Data
Mar. 23, 2007 (GB) 0705617.9

(51) Int. Cl. A61B 17/00; A61B 19/00; A61B 17/58

(52) U.S. Cl. 606/80; 128/898; 606/96

(57) ABSTRACT

A method of bone surgery including drilling a first bore in a near cortex of the bone with a first drill bit. Then a second drill bit is passed though the first bore towards a far cortex of the bone, the second drill bit comprising a distal shaft portion and a proximal shaft portion, the distal shaft portion having a diameter less than a diameter of the proximal shaft portion such that a shoulder is defined between the distal and proximal shaft portions. Then a second bore is drilled in the far cortex of the bone with the distal shaft portion of the second drill bit whilst the second drill bit is received in the first bore.

### CROSS-REFERENCE TO RELATED APPLICATION

This application claims priority to U.K. Patent Application No. 0705617.9, filed Mar. 23, 2007, which is incorporated herein by reference.

### FIELD OF THE INVENTION

The present invention relates to a method of bone surgery.

### BACKGROUND TO THE INVENTION

A fractured bone may have a form in which two bone portions define a fracture between them. Muscles and ligaments around the bone can cause the bone portions to be placed under compression or extension causing the relative disposition of the bone portions to change. Upon healing of the fracture such a change in disposition can result in a loss of the original bone shape, i.e. the shape before the fracture. Accordingly, it is known to hold the bone portions in relation to each other by means of a bone screw, which compresses the fractured bone portions. The bone screw maintains the bone portions in a desired relative disposition until the fracture heals and thereby reduces the likelihood of loss of the original bone shape.

The bone screw is brought into engagement with the bone portions by drilling through the bone cortices to form bores and such that the bores are at a desired angle in relation to each other in accordance with the configuration of the fracture. Threaded portions of the screw engage with the bores.

### SUMMARY OF THE DISCLOSURE

The present inventor has appreciated shortcomings of known methods of forming bores in the cortices of a bone during bone fixing procedures.

Thus, it is an object for the present disclosure to provide an improved method of bone surgery in which a bore is formed in each of opposing cortices of a bone having a fracture.

It is a further object for the present disclosure to provide a method of bone surgery in which a bore is formed in at least one bone cortex by means of a drill.

According to a first aspect of the disclosure there is provided a method of bone surgery, the method comprising:

drilling a first bore in a near cortex of the bone with a first drill bit;

passing a second drill bit though the first bore towards a far cortex of the bone, the second drill bit comprising a distal shaft portion and a proximal shaft portion, the distal shaft portion having a diameter less than a diameter of the proximal shaft portion such that a shoulder is defined between the distal and proximal shaft portions; and

drilling a second bore in the far cortex of the bone with the distal shaft portion of the second drill bit whilst the second drill bit is received in the first bore.

Typically, the method of the present disclosure is used to fix two portions of a bone, the two portions defining a fracture between them. Thus, the first bore is formed in the near cortex of the first bone portion and the second bore is formed in the far cortex of the second bone portion. A surgeon drills from a first side of a bone though to a second side of the bone and thus the near cortex is taken to be at the first side of the bone and the far cortex is taken to be at the second side of the bone. After practicing the method according to the present invention, a screw may be received through and engage with the first bore and be received in and engage with the second bore such that the screw spans the fracture and is securely attached to the two bone portions. Thus, the screw can be used to hold a relative disposition of the two bone portions.

Drilling the second bore in the far cortex of the bone according to the invention reduces the risk of the second drill bit advancing too far into the far cortex. For example, there may be vital structures, such as neurovascular structures, beyond the exterior surface of the far cortex and there is a risk of a second drill bit of known type having a body of uniform diameter advancing during drilling of the second bore beyond the exterior surface of the far cortex and damaging the vital structures. This is because a surgeon normally applies force to the second drill bit to cause it to advance into the far cortex. When the surgeon is applying force it is not unknown for resistance to reduce, e.g. as may be caused by a collapse of bone structure in the immediate vicinity of the tip of the drill bit, causing over advancement of the drill bit. However, if over advancement happens during practice of the present invention, the shoulder of the second drill abuts against the interior surface of the far cortex and arrests the advancement to thereby prevent or at least reduce the likelihood of damage to the vital structures.

In addition, a length of the distal shaft portion may be predetermined to determine an extent to which the second drill bit advances through the far cortex. This is because advancement of the second drill bit is arrested when the shoulder abuts against the interior surface of the far cortex such that only the distal shaft portion is received by the far cortex. Thus, there may be no need for the surgeon to carefully control an extent to which the drill is advanced into the far cortex as may be required with a drill bit of uniform diameter according to known practice. The length of the second drill bit from the shoulder to a location on the proximal shaft portion extending beyond the exterior surface of the near cortex may be used to determine a length of bone screw to be used. More specifically, markings, e.g. on the proximal shaft portion with respect to a reference point on another component, such as a drill sleeve, may be used to indicate an appropriate length of screw to be used.

More specifically, the step of drilling a second bore in a far cortex of the bone may consist of forming a blind bore in the far cortex. The length of the distal shaft portion may determine the depth of the blind bore from the interior surface of the far cortex.

Alternatively or in addition, the method may further comprise removing the first drill bit from the first bore before the step of passing the second drill bit though the first bore.

Alternatively or in addition, the proximal shaft portion may have a diameter such the second drill bit is a snug fit in the first bore when the step of passing the second drill bit through the first bore is performed.

More specifically, the diameter of the proximal shaft portion may be substantially the same as a diameter of the first drill bit that formed the first bore.

Where the second drill bit is a snug fit in the first bore the second drill bit may be used to hold a portion of the bone in which the first bore has been formed in relation to a portion of the bone in which the second bore has been formed. Thus, the second drill bit can function not only as a drill bit per se but also as a device that holds bone portions in relation to each other.

Hence, the method may further comprise maintaining the second drill bit such that it is received in the first and second bores whilst a further procedural step is performed, such as engaging a bone fixing apparatus with the second drill bit. For example, bone fixing apparatus according to WO 2005/020831 may be brought into engagement with the second drill bit. Thus, the second drill bit can be used to define a plane in which a bone screw will subsequently be located in the bone, with a longitudinal axis of the second drill bit being parallel to a longitudinal axis of the bone screw.

More specifically, the second drill bit may be maintained such that a portion of the second drill bit extends beyond an exterior surface of the near cortex by a distance of at least substantially 40 mm. A distance of at least substantially 40 mm has been found to be required for attachment of apparatus thereto.

More specifically, the second drill bit may extend beyond an exterior surface of the near cortex by a distance of at least substantially 60 mm.

More specifically, the second drill bit may extend beyond an exterior surface of the near cortex by a distance of between substantially 100 mm and substantially 150 mm. A distance of between substantially 100 mm and substantially 150 mm has been found to cater for engagement with typical apparatus at various angles of the drill bit in relation to the bone and with different bone sizes.

Alternatively or in addition, the step of drilling the first bore in a near cortex may be performed with a first drill bit having a distal shaft portion and a proximal shaft portion, the distal shaft portion having a diameter less than a diameter of the proximal shaft portion such that a shoulder is defined between the distal and proximal shaft portions.

More specifically, the method may comprise advancing the distal shaft portion of the first drill bit into the near cortex to an extent that the shoulder abuts against an exterior surface of the near cortex. Thus, the first drill bit may advance into the bone no further than a length of the distal shaft portion to thereby, for example, reduce the likelihood of damage to the far cortex if the first drill bit becomes over advanced.

Alternatively or in addition, the proximal shaft portion of the second drill bit and the distal shaft portion of the first drill bit may have diameters such that the second drill bit is a snug fit in the first bore.

More specifically, the diameter of the proximal shaft portion of the second drill bit may be substantially the same as the diameter of the distal shaft portion of the first drill bit.

Alternatively or in addition, at least one of the first drill bit and the second drill bit may define a bore. The bore may, for example, receive a guide wire on which the drill bit is run to provide for proper location of a bore being drilled in the bone. A guide wire may be required where precise location of a bore is desired, e.g. in a small bone, such as the distal radius, or where the bone is being fixed by an operator of lesser skill.

Alternatively or in addition, at least one of the first drill bit and the second drill bit may have at least one cutting blade. More specifically, a cutting blade may be one of linear, helical and trocar in form.

Alternatively or in addition, at least one of the first drill bit and the second drill bit may have at least one cutting surface, the cutting surface being non-planar in profile. Such a configuration may find use where the second drill bit is of narrow gauge and a bore through the second drill bit is of comparatively wide diameter such that no point is defined by a leading edge of the distal shaft portion of the second drill bit. Thus, the cutting surface may be comprised in a portion of a leading edge of the proximal shaft portion around the bore opening.

More specifically, the cutting surface may define a plurality of protrusions, which may be substantially evenly spaced apart from each other. More specifically, the protrusions may be angular in profile. Thus, the cutting surface may define teeth. Alternatively or in addition, the method may further comprise disposing at least one drill guide adjacent a surface of a cortex to be drilled. For example, the drill guide may be disposed such that a first end abuts an exterior surface of the near cortex and the first drill may be received in the second, opposing end of the drill guide before the step of drilling the first bore is performed.

More specifically, the method may comprise disposing each of a plurality of drill guides of different diameters in turn adjacent a surface of the cortex to be drilled. The plurality of drill guides may be concentrically disposed, as described in WO 2005/020831, and brought into use from a disposition in which one drill guide is nested within another.

More specifically, the plurality of drill guides may be brought into use out of order with respect to their respective diameters. For example, a narrow gauge drill guide may be brought into use when a bore is being drilled for a guide wire. Then a wide gauge drill guide may be brought into use when the first bore is being drilled with the first drill. Then a medium gauge drill guide may be brought into use when the second bore is being drilled with the second drill. Then a drill guide of wider gauge than the drill guide for the first drill may be brought into use for introduction of the bone screw.

According to a second aspect of the present disclosure there is provided a method of bone surgery, the method comprising: drilling a first bore in a near cortex of the bone with a first drill bit; passing a second drill bit though the first bore towards a far cortex of the bone; and drilling a second bore in the far cortex of the bone with the second drill bit while the second drill bit is received in the first bore. Embodiments of the second aspect of the present disclosure may comprise one or more features of the first aspect of the present disclosure.

It should be appreciated that the present invention can be implemented in numerous ways, including as a process, an apparatus, a system, a device, a method for applications now known and later developed. These and other features of the system disclosed herein will become more readily apparent from the following description and the drawings.

### BRIEF DESCRIPTION OF THE DRAWINGS

The present invention will now be described by way of example only with reference to the following drawings, of which:

FIG. 1 is a representation of a bone having a fracture in which a bone screw is used to fix the bone according to the present disclosure;

FIG. 2 is a schematic view of a first drill bit for use in the present disclosure;

FIG. 3 is a schematic view of a second drill bit for use in the present disclosure;

FIG. 4 is a perspective view of bone fixing apparatus being used with the second drill bit;

FIG. 5 is a view of the bone screw of FIG. 1; and

FIG. 6 is flow chart showing the steps involved in the present disclosure.

### DETAILED DESCRIPTION OF PREFERRED EMBODIMENTS

The advantages, and other features of the system disclosed herein, will become more readily apparent to those having ordinary skill in the art from the following detailed description of certain preferred embodiments taken in conjunction with the drawings which set forth some representative embodiments of the present invention. The apparatus used in the present disclosure will be described before the method of the disclosure is described.

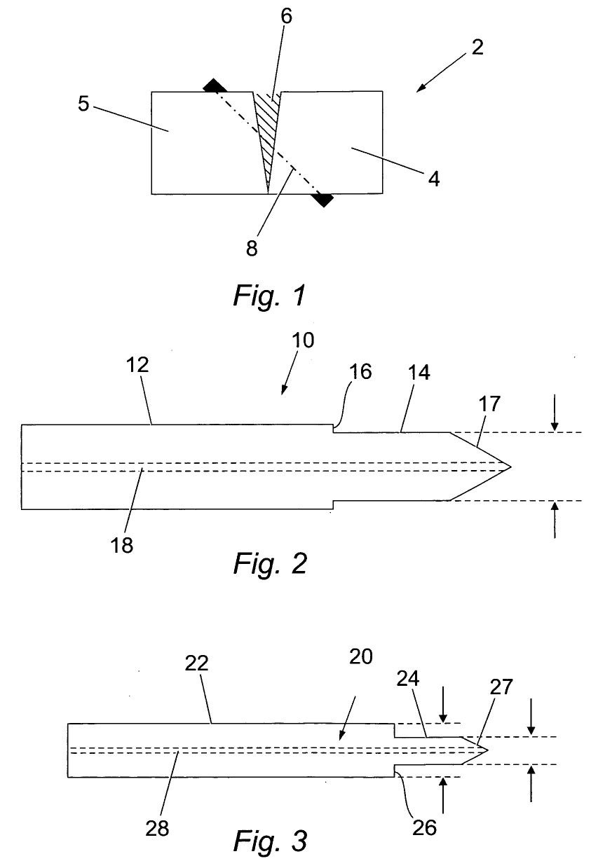

As described above, a fractured bone 2 may have the form shown in FIG. 1, in which two bone portions 4, 5 define a fracture 6 between them. Muscles and ligaments around the bone can cause the bone portions 4, 5 to be placed under compression or extension causing the relative disposition of the bone portions to change. Upon healing of the fracture such a change in disposition can result in a loss of the original bone shape. According to the method of the present disclosure, bores are drilled in the bone portions 4, 5 to receive and engage with the bone screw 8. As can be seen from FIG. 1, a first end of the bone screw 8 engages with one of the bone portions and a second, opposing end of the bone screw engages with the other of the bone portions with a body of the bone screw bridging the fracture. The bone screw maintains the bone portions in a desired relative disposition until the fracture heals and thereby reduces the likelihood of loss of the original bone shape.

The bone screw 8 of FIG. 1 is brought into engagement with the bone portions 4, 5 by drilling through the bone cortices to form bores and such that the bores are at a desired angle in relation to each other in accordance with the configuration of the fracture. Threaded portions of the bone screw 8 engage with the bores.

FIG. 2 provides a schematic view of a first drill bit 10 used in the disclosure. The first drill bit has a proximal shaft portion 12, which is gripped by a drill of known type used in surgical procedures, and a distal shaft portion 14. The distal shaft portion 14 has a smaller diameter than the proximal shaft portion 12 such that a shoulder is 16 defined between them. The leading edge 17 of the distal shaft portion defines two linear cutting blades that converge away from the proximal shaft portion 12 towards a point. The first drill bit 10 defines an open ended bore 18 that extends linearly from the point defined by the two cutting blades to a rear-most face of the proximal shaft portion 12.

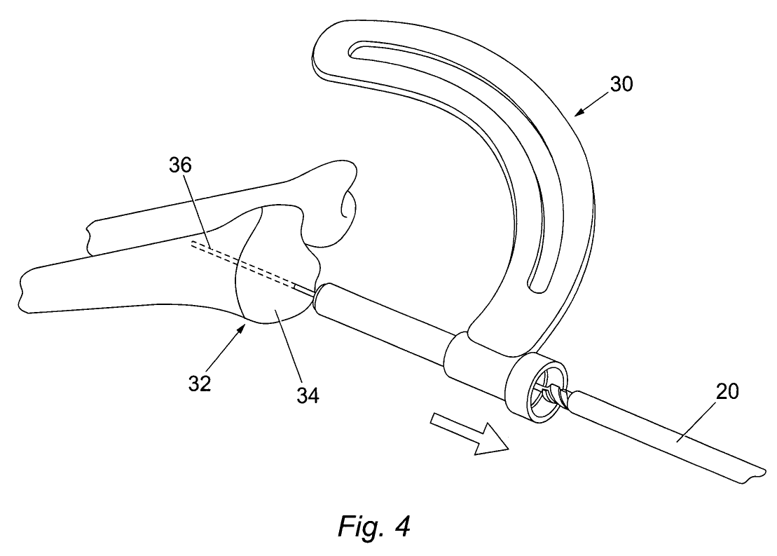

FIG. 3 provides a schematic view of a second drill bit 20 used in the disclosure. The second drill bit has a proximal shaft portion 22, which is gripped by the drill mentioned in the immediately preceding paragraph, and a distal shaft portion 24. The distal shaft portion 24 has a smaller diameter than the proximal shaft portion 22 such that a shoulder is 26 defined between them. The leading edge 27 of the distal shaft portion defines two linear cutting blades that converge away from the proximal shaft portion 22 towards a point. The second drill bit 20 defines an open ended bore 28 that extends linearly from the point defined by the two cutting blades to a rear-most face of the proximal shaft portion 22. A diameter of the proximal shaft portion 22 of the second drill bit 20 and a diameter of the distal shaft portion 12 of the first drill bit 10 are substantially the same. Although not reflected in the relative dimensions of the drill bits shown in FIGS. 2 and 3, the second drill bit 20 is longer than the first drill bit 10 such that the proximal shaft portion of the second drill bit extends beyond an exterior surface of a near cortex of a bone as is described below in more detail.

In an un-illustrated form of the second drill bit shown in FIG. 3, the distal shaft portion 24 is narrower and the bore 27 wider such that the leading edge 27 is significantly truncated. In such a form, a cutting surface is defined in the portion of the distal shaft portion surrounding the bore 27. The cutting surface comprises a plurality of substantially evenly spaced apart teeth that extend forwards of the bore opening. Such a form is often preferred where the truncation of the leading edge 27 is such that there is insufficient space to form a cutting blade of linear, helical or trocar form.

FIG. 4 shows a perspective view of bone fixing apparatus 30 in use with the second drill bit 20 of FIG. 3. The form and function of the bone fixing apparatus 30 is described in detail in WO 2005/020831. In FIG. 4 the bone fixing apparatus 30 and the second drill bit 20 are shown being used to fix the distal radius 32. The near cortex 34, i.e. the cortex that receives the drill first, and the far cortex 36, i.e. the cortex that receives the drill after it has been received by the near cortex, are shown in FIG. 4.

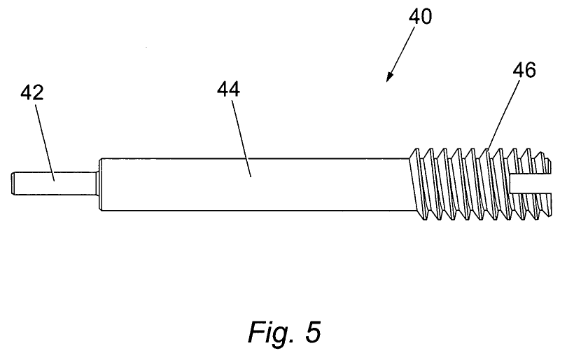

The bone screw 40 of FIG. 5 has a distal screw portion 42 and main body 44. The proximal end of the main body defines a thread 46.

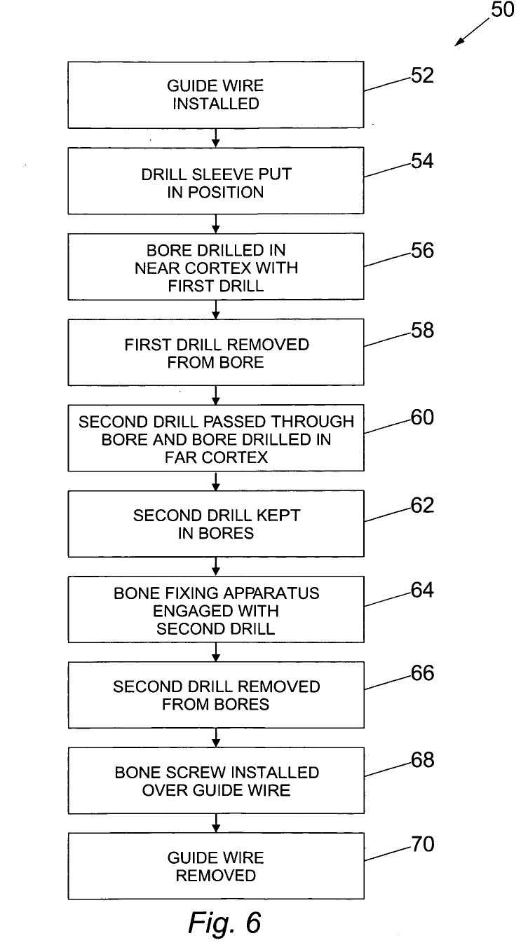

The method of bone surgery according to the present disclosure will now be described with reference to the flow chart 50 of FIG. 6. After preparation of the area around the bone to be treated, a drill is used in conjunction with a narrow gauge drill guide to form a narrow diameter open ended bore from the near cortex to the far cortex of the bone such that the ends of the bore are at the locations where the bone screw 40 of FIG. 5 is to engage. In accordance with known practice an end of a guide wire is passed though a protective sleeve of narrow gauge and the bore before being anchored 52 to provide a means to run bone fixing apparatus and to hold the bone portions while the bone fixing apparatus is brought into use. Then and in accordance with known practice a drill sleeve (not shown) 54 of wide gauge is run over the guide wire such that one end of the drill sleeve abuts an exterior surface of the near cortex 34 of the bone 32. The first drill bit 10 is then used to drill a first bore 56 in the near cortex 34 with the shoulder 16 preventing more than the distal shaft portion 14 from advancing into the bone. The first drill bit 10 is withdrawn from the formed first bore 58 and the second drill bit 20 passed through a drill guide of medium gauge and the first bore towards the interior surface of the far cortex 60. The second drill bit 20 is used to form a blind, second bore 60 in the far cortex 36 with the shoulder 26 preventing more than the distal portion 24 of the second drill bit 20 from advancing into the far cortex. The length of the distal portion 24 defines the depth of the blind, second bore. The length of the second drill bit 20 is chosen such that between substantially 100 mm and 150 mm of the second drill bit extends beyond the exterior surface of the near cortex, having regards to the separation of the locations on the first and second bone portions 4, 5 that will engage with the bone screw and the configuration of the bone fixing apparatus 30 to be brought into engagement with the second drill bit.

The second drill bit 20 is maintained such that it is received securely in the first and second bores 62. The diameter of the proximal portion 22 of the second drill bit is such that it is a snug fit in the first bore to thus allow the second drill bit 20 to hold the first and second bone portions 4, 5 in the desired relative disposition. Next the bone fixing apparatus 30 of FIG. 4 is run over the second drill bit 20 such that the precision of location provided by the second drill bit is transferred to the bone fixing apparatus 64. As described in WO 2005/020831 the bone fixing apparatus 30 may be used for precise location of second and further bone screws, with the precision of location being achieved by reference to the first located screw. Thus, the second drill bit performs the important function of providing a rigid platform from which precision of location of second and further bone screws can be achieved. The second drill bit is then removed 66 before the bone screw 40 is run into the bore of the bone fixing apparatus on the guide wire and passed through the first bore 68. The bone screw is advanced into the bone to the extent that the distal screw portion 42 is received in the second, blind bore and the threaded portion 46 engages with the first bore 68 to thereby arrive at the bone fixing configuration shown in FIG. 1. Then the guide wire is removed 70. The second drill bit 20 thus defines with the guide wire the axis on which the bone screw 40 is subsequently run when the bone screw is being brought into engagement with the first and second bores.

Unless otherwise specified, the illustrated embodiments can be understood as providing exemplary features of varying detail of certain embodiments, and therefore, unless otherwise specified, features, components, modules, elements, and/or aspects of the illustrations can be otherwise combined, interconnected, sequenced, separated, interchanged, positioned, sized, and/or rearranged without materially departing from the disclosed systems or methods. Additionally, the shapes and sizes of components are also exemplary and can be altered without materially affecting or limiting the disclosed technology. While the invention has been described with respect to preferred embodiments, those skilled in the art will readily appreciate that various changes and/or modifications can be made to the invention without departing from the spirit or scope of the invention as defined by the claims.

What is claimed is:

1. A method of bone surgery, the method comprising:
drilling a first bore in a near cortex of a bone with a first drill bit;
passing a second drill bit though the first bore and towards a far cortex of the bone, the second drill bit comprising a distal shaft portion and a proximal shaft portion, the distal shaft portion having a diameter less than a diameter of the proximal shaft portion such that a shoulder is defined between the distal and proximal shaft portions; and
drilling a second bore in the far cortex of the bone with the distal shaft portion of the second drill bit whilst the second drill bit is received in the first bore.

2. The method according to claim 1, in which the step of drilling the second bore in the far cortex of the bone consists of forming a blind bore in the far cortex.

3. The method according to claim 1, in which the method further comprises removing the first drill bit from the first bore before the step of passing the second drill bit though the first bore.

4. The method according to claim 1, in which the proximal shaft portion has a diameter such the second drill bit is a snug fit in the first bore when the step of passing the second drill bit through the first bore is performed.

5. The method according to claim 4, in which the diameter of the proximal shaft portion is substantially the same as a diameter of the first drill bit that formed the first bore.

6. The method according to claim 4, in which the method further comprises maintaining the second drill bit such that the second drill bit is received in the first and second bores whilst a further step of a bone fixing procedure is performed.

7. The method according to claim 6 in which the second drill bit is maintained such that a portion of the second drill bit extends beyond an exterior surface of the near cortex by a distance of at least substantially 40 mm.

8. The method according to claim 7, in which the second drill bit extends beyond an exterior surface of the near cortex by a distance of at least substantially 60 mm.

9. The method according to claim 8, in which the second drill bit extends beyond an exterior surface of the near cortex by a distance of between substantially 100 mm and substantially 150 mm.

10. The method according to claim 1, in which the step of drilling the first bore in a near cortex is performed by a distal shaft portion of a first drill bit, the first drill bit also having a proximal shaft portion, the distal shaft portion having a diameter less than a diameter of the proximal shaft portion such that a shoulder is defined between the distal and proximal shaft portions.

11. The method according to claim 10, in which the method comprises advancing the distal shaft portion of the first drill bit into the near cortex to an extent that the shoulder abuts against an exterior surface of the near cortex.

12. The method according to claim 10, in which the proximal shaft portion of the second drill bit and the distal shaft portion of the first drill bit have diameters such that the second drill bit is a snug fit in the first bore.

13. The method according to claim 12, in which the diameter of the proximal shaft portion of the second drill bit is substantially the same as the diameter of the distal shaft portion of the first drill bit.

14. The method according to claim 1, further comprising running at least one of the first drill bit and the second drill bit on a guide wire, the bit defining a bore, which receives the guide wire.

15. The method according to claim 1, in which at least one of the steps of drilling the first and second bores is performed by a drill bit having at least one cutting blade, the cutting blade being one of linear, helical and trocar in form.

16. The method according to claim 1, in which at least one of the first drill bit and the second drill bit has at least one cutting surface, the cutting surface being non-planar in profile.

17. The method according to claim 16, in which the cutting surface defines a plurality of protrusions.

18. The method according to claim 17, in which the protrusions are angular in profile.

19. The method according to claim 1, in which the method further comprises disposing at least one drill guide adjacent a surface of a cortex to be drilled.

20. The method according to claim 19, in which the method comprises disposing each of a plurality of drill guides of different diameters in turn adjacent a surface of a cortex to be drilled.

21. The method according to claim 20, in which the plurality of drill guides are brought into use out of order with respect to their respective diameters.
